(19) United States
(12) Patent Application Publication
(10) Pub. No.: US 2024/0424226 A1
(43) Pub. Date: Dec. 26, 2024

(54) VENTED SYRINGE ADAPTER WITH BULB

(71) Applicant: CareFusion 303, Inc., San Diego, CA (US)

(72) Inventors: Suhas Hanumantharao, Bengaluru (IN); Arjun Murali, Bangalore (IN); Arun Karthik Senthil Murugan, Bangalore (IN); Kanjimpuredathil Muralikrishna Menon, Bangalore (IN); Rahul Rajan Puthukkad, Bengaluru (IN)

(21) Appl. No.: 18/338,497

(22) Filed: Jun. 21, 2023

(51) Int. Cl. A61M 5/34; A61M 5/31; A61M 5/315; A61M 39/10; A61M 39/24

(52) U.S. Cl. CPC A61M 5/345; A61M 5/3129; A61M 5/31511; A61M 39/10; A61M 39/24

(57) ABSTRACT

Syringe adapters are described herein. A syringe adapter includes an adapter body, a coupling portion, and a bulb. The adapter body defines a fluid channel in fluid communication with the syringe and configured to permit medical fluid flow from the syringe through the syringe adapter and a vent channel in fluid communication with the syringe and configured to permit air flow into the syringe. The coupling portion defines an adapter portion configured to be coupled to the adapter body and a syringe portion configured to be coupled to the syringe. The bulb is coupled to the adapter body and defines a bulb volume that is in fluid communication with the fluid channel and is movable between a collapsed configuration configured to displace air from the bulb volume and the fluid channel and an expanded configuration configured to draw medical fluid from the syringe into the fluid channel and bulb volume.

### FIELD OF THE INVENTION

The present disclosure relates generally to medical connectors used in fluid transfer applications. More particularly, the present disclosure relates to a syringe adapter for the transfer of fluids in medical settings.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag or a syringe. In some applications, infusion delivery can take place with other suitable devices or arrangements.

Unlike other fluid sources, such as collapsible bags, the fluid-filled syringe includes a rigid structure and cannot collapse when the plunger of the syringe is stationary and the fluid is being pulled from the syringe. As a result, ambient air needs to be introduced into the syringe to replace the fluid that is being pulled from the syringe.

### SUMMARY

The disclosed subject matter relates to syringe adapters. In certain embodiments, a syringe adapter for coupling with a syringe is disclosed that comprises an adapter body defining a fluid channel in fluid communication with the syringe and configured to permit medical fluid flow from the syringe through the syringe adapter and a vent channel in fluid communication with the syringe and configured to permit air flow into the syringe; a coupling portion defining an adapter portion configured to be coupled to the adapter body and a syringe portion configured to be coupled to the syringe; and a bulb coupled to the adapter body and defining a bulb volume, wherein the bulb volume is in fluid communication with the fluid channel and is movable between a collapsed configuration configured to displace air from the bulb volume and the fluid channel and an expanded configuration configured to draw medical fluid from the syringe into the fluid channel and bulb volume.

In certain embodiments, a method to prime an IV set is disclosed that comprises coupling a syringe to a syringe adapter; displacing air from a fluid channel of the syringe adapter into the syringe by collapsing a bulb coupled to the syringe adapter; and drawing medical fluid from the syringe into the fluid channel of the syringe adapter by expanding the bulb.

In certain embodiments, an IV set is disclosed that comprises a syringe comprising: a barrel; and a plunger movably disposed in the barrel, wherein the plunger and the barrel cooperatively defining a syringe volume; and a syringe adapter comprising: an adapter body defining a fluid channel in fluid communication with the syringe volume and configured to permit medical fluid flow from the syringe volume through the syringe adapter and a vent channel in fluid communication with the syringe volume and configured to permit air flow into the syringe volume; a coupling portion defining an adapter portion coupled to the adapter body and a syringe portion coupled to the syringe; and a bulb coupled to the adapter body and defining a bulb volume, wherein the bulb volume is in fluid communication with the fluid channel and the syringe volume and is movable between a collapsed configuration configured to displace air from the bulb volume and the fluid channel and an expanded configuration configured to draw medical fluid from the syringe volume into the fluid channel and bulb volume.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates a patient receiving an infusion of a medical fluid according to certain aspects of the present disclosure.

FIG. 2 illustrates a front view of a syringe assembly according to certain aspects of the present disclosure.

FIG. 3 illustrates a cross-sectional view of the syringe assembly of FIG. 2.

FIG. 4 illustrates a cross-sectional view of the syringe adapter of FIG. 2.

FIG. 5 illustrates the syringe adapter of FIG. 2 with the bulb of the syringe adapter in a collapsed configuration.

FIG. 6 illustrates the syringe adapter of FIG. 2 with the bulb of the syringe adapter in an expanded configuration.

FIG. 7 illustrates the syringe adapter of FIG. 2 with the bulb of the syringe adapter in a primed configuration.

### DETAILED DESCRIPTION

The disclosed syringe adapter incorporates a bulb in fluid communication with the fluid channel. The bulb can be deformable to displace air from the bulb and fluid channel and draw medical fluid from the syringe into the bulb and fluid channel. By utilizing a bulb to displace air and draw in medical fluid, the process or workflow to prime the syringe adapter and IV set can be simplified.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to the filters for the administration of medical fluid using the disclosed syringe adapter, it is to be understood that this description is only an example of usage and does not limit the scope of the claims. Various aspects of the disclosed filter may be used in any application where it is desirable to prime a syringe adapter with a simplified process.

The disclosed syringe adapter overcomes several challenges discovered with respect to certain conventional syringe adapters. One challenge with certain conventional syringe adapters is that certain conventional syringe adapters are intended to be used with infusion pumps and are not compatible with gravity infusions. Further, another challenge with certain conventional syringe adapters is that certain conventional syringe adapters require a time consuming or complex workflow to prepare and/or prime the IV for infusion. For example, a health care professional may be required to connect a syringe to the conventional syringe adapter, connect a saline line, aspire the syringe and prime the conventional syringe adapter in an upstream fluid flow, disconnect the saline line, transport the conventional syringe adapter from the preparation space to the medical delivery space, connect the medication syringe, and connect the patient end of the IV set. Because a health care professional is required to perform numerous steps in preparation for an infusion, utilizing a conventional syringe adapter may introduce delays and or errors during the process.

Therefore, in accordance with the present disclosure, it is advantageous to provide a syringe adapter as described herein that allows for gravity infusion from a syringe and reduces the time and workflow steps required to prepare and/or prime the syringe adapter. Further, it is advantageous to provide a syringe adapter that is familiar and straightforward for a healthcare professional to operate.

Examples of syringe adapters that allow for gravity infusions from syringes and a simplified workflow are now described.

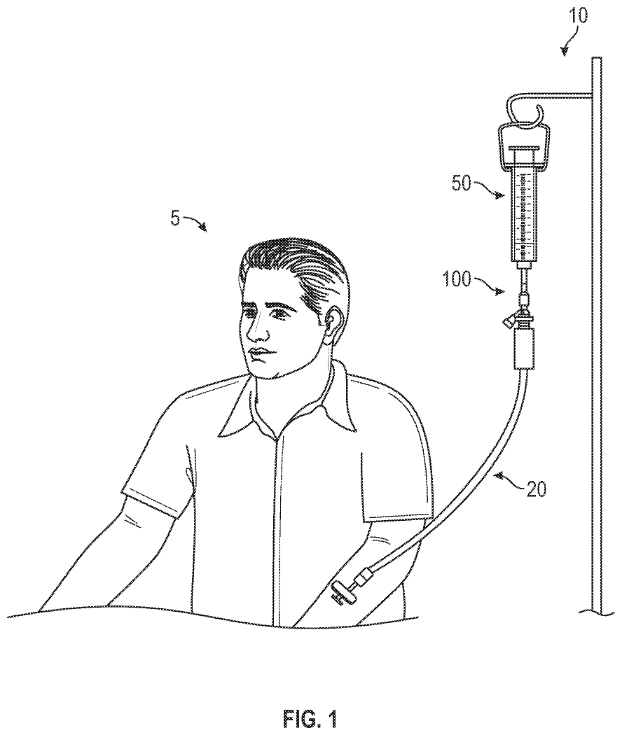

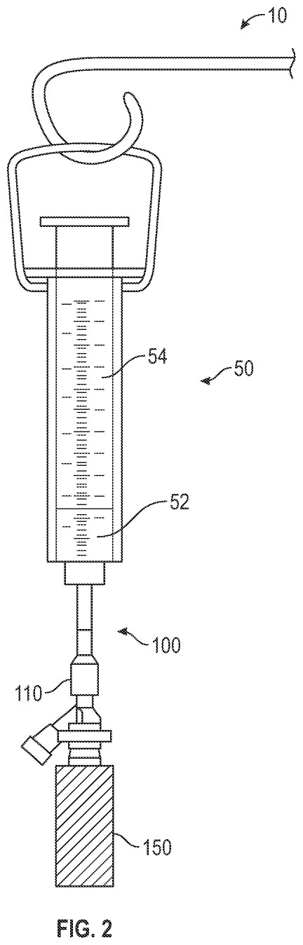

FIG. 1 illustrates a patient 5 receiving an infusion of a medical fluid according to certain aspects of the present disclosure. FIG. 2 illustrates a front view of a syringe assembly 10 according to certain aspects of the present disclosure. With reference to FIGS. 1 and 2, a syringe assembly 10 delivers a gravity infusion of medical fluid from a syringe 50 to the patient 5. As illustrated, medical fluid from the syringe assembly 10 is delivered to the patient 5 via the IV set 20. In the depicted example, the syringe assembly 10 includes a syringe adapter 100 that facilitates the connection and flow of medical fluid from the syringe 50 to the IV set 20 and to the patient 5.

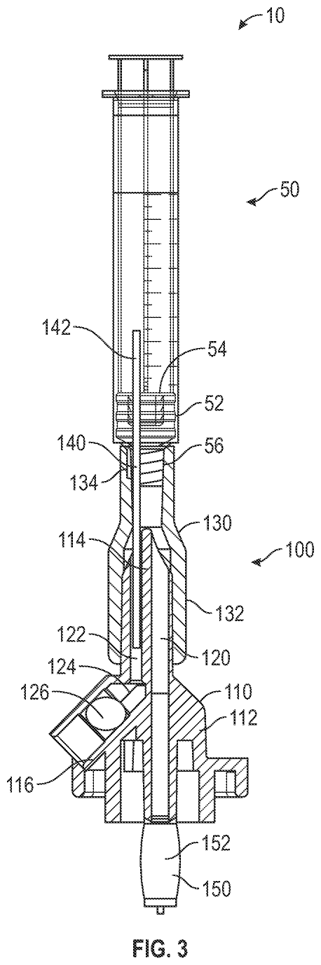

As illustrated, the syringe 50 can store and dispense medical fluid. The syringe 50 includes a syringe barrel 52 and a plunger 54 movably disposed within the syringe barrel 52. The syringe 50 can store medical fluid in the syringe volume defined by the syringe barrel 52 and the plunger 54. During operation, the plunger 54 can be retracted to draw medical fluid into the syringe barrel 52 and advanced to dispense or administer medical fluid from the syringe barrel 52. In some embodiments, the end or tip 56 of the syringe 50 can include a Luer connector (as shown in FIG. 3).

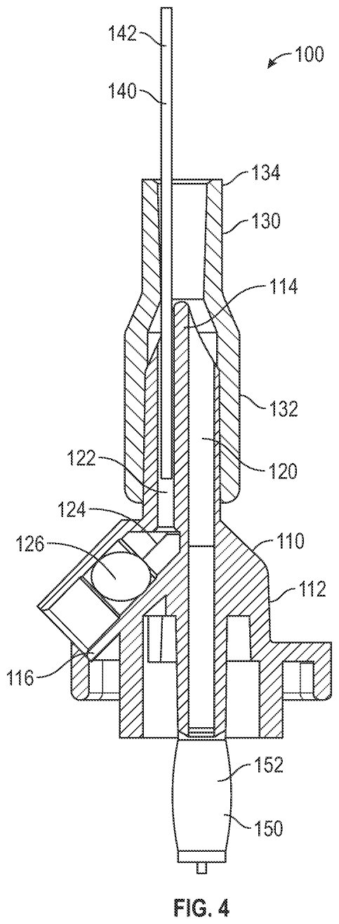

FIG. 3 illustrates a cross-sectional view of the syringe assembly 10 of FIG. 2. FIG. 4 illustrates a cross-sectional view of the syringe adapter 100 of FIG. 2. In the depicted example, the syringe adapter 100 can facilitate a physical connection or coupling between the syringe 50 and the IV set. As illustrated, the syringe adapter 100 can include a coupling portion 130 that couples the syringe 50 to the adapter body 110. In some embodiments, a syringe portion 134 of the coupling portion 130 can couple to an end or tip 56 of the syringe 50. Optionally, the syringe portion 134 of the coupling portion 130 can include a mating Luer connector to engage with the Luer connector of the syringe 50.

As illustrated, an adapter portion 132 disposed at the opposite end of the coupling portion 130 can couple to the adapter body 110 of the syringe adapter 100. In some embodiments, the adapter portion 132 can be disposed over a portion of the adapter body 110. In some embodiments, the adapter portion 132 can couple to a spike portion 114 of the adapter body 110 or any other suitable portion of the adapter body 110.

Further, the adapter body 110 can be coupled to a portion of the IV set. In the depicted example, the bulb 150 coupled to the adapter body 110 may be coupled or otherwise attached to the IV set. In some embodiments, a suitable portion of the adapter body 110 opposite to the spike portion 114 can be coupled to the IV set.

In the depicted example, the syringe adapter 100 allows for fluid flow from the syringe 50 to the IV set. As illustrated, the adapter body 110 defines a fluid channel 120 that provides fluid communication between the syringe 50 and the IV set. In some embodiments, the fluid channel 120 extends along a length of the adapter body 110. The fluid channel 120 may extend along a length of the spike portion 114 and into the lower portion 112 of the adapter body 110. The fluid channel 120 can be disposed along or near a central axis of the adapter body 110. Further, the coupling portion 130 can further facilitate fluid flow between the syringe 50 and an upper portion of the fluid channel 120.

As illustrated, the fluid channel 120 permits flow from the adapter body 110 to the IV set coupled to the adapter body 110. A lower portion of the fluid channel 120 can be connected to or otherwise in fluid communication with the IV set. As illustrated, the fluid channel 120 can be in fluid communication with a bulb 150, which is in turn connected to or otherwise in fluid communication with the IV set.

As described above, in some applications, the syringe 50 includes a rigid structure and cannot collapse when the plunger 54 of the syringe 50 is stationary. Therefore, during a gravity infusion or other administration of medical fluid from the syringe 50, ambient air must be introduced into the syringe 50 to replace the medical fluid dispensed from the syringe 50. In the depicted example, the syringe adapter 100 allows for air to flow into the syringe 50 to displace medical fluid that is dispensed during fluid administration. As illustrated, the adapter body 110 defines a vent channel 122 that provides fluid communication between the syringe 50 and the ambient air or environment surrounding the syringe assembly 10. In some embodiments, the vent channel 122 extends along a portion of the length of the adapter body 110. The vent channel 122 may extend along a length of the spike portion 114 and partially into the lower portion 112 of the adapter body 110.

As illustrated, the vent channel 122 permits environmental or ambient air to enter the adapter body 110 and be directed to the syringe 50. The outer or lower portion 124 of the vent channel 122 is in fluid communication with the environment or ambient surroundings. In the depicted example, the lower portion 124 of the vent channel 122 can include a one-way or check valve 126 to allow airflow into the vent channel 122 and prevent the flow of medical fluid from the syringe 50 out of the vent channel 122. The check valve 126 can include a ball mechanism to allow airflow into the vent channel 122 and prevent leakage or outflow of medical fluid through the vent channel 122.

In some embodiments, a cannula 140 can extend between the vent channel 122 and the syringe 50 to allow environmental or ambient air from the vent channel 122 a direct path to enter the syringe 50 and displace the fluid being dispensed. In the depicted example, a portion of the cannula 140 extends into the vent channel 122 and an opposite end or portion of the cannula 140 extends into the syringe 50. The cannula lumen 142 of the cannula 140 can be in fluid communication with the vent channel 122 and the syringe 50 to facilitate air flow from the environment into the syringe 50 via the vent channel 122. As illustrated, the cannula 140 can extend across the coupling portion 130 of the syringe adapter 100. Advantageously, by providing a direct path for fluid communication between the syringe 50 and the environment via the cannula 140, the syringe adapter 100 can allow for air to rapidly enter the syringe 50 as medical fluid is dispensed or administered from the syringe 50. In contrast, certain conventional syringe adapters may allow for air entering the syringe to slowly pass-through medical fluid before reaching the top of the syringe volume.

In the depicted example, the syringe adapter 100 can include a bulb 150 to allow for rapid priming of the syringe adapter 100 and the IV set. In some embodiments, the bulb 150 is formed from a resilient or deformable material that allows the bulb volume 152 defined by the bulb 150 to be reduced upon actuation. As illustrated, the bulb volume 152 is in fluid communication with the fluid channel 120. In some embodiments, the bulb 150 is disposed at a lower end of the syringe adapter 100. The bulb 150 can be formed from any resilient or deformable material, including, but not limited to silicone, rubber, or thermoplastic elastomers. The bulb 150 can have a generally cylindrical shape. In some embodiments, the bulb 150 may include or define a drip former.

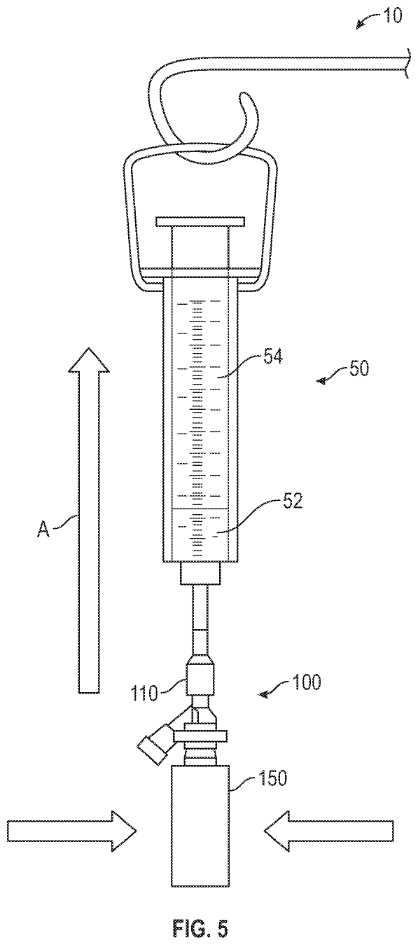

After connecting the syringe 50 to the syringe adapter 100, the syringe adapter 100 can be primed. During priming, the bulb 150 can expel air from the syringe adapter 100 and draw in fluid from the syringe 50 and into the syringe adapter. In some embodiments, an auto priming cap can be utilized with the syringe 50. FIG. 5 illustrates the syringe adapter 100 of FIG. 2 with the bulb 150 of the syringe adapter in a collapsed configuration. With reference to FIG. 5, a healthcare professional can compress, squeeze, or collapse the bulb 150 to reduce the bulb volume 152, expelling air A in the bulb volume 152 and the fluid channel 120 upward toward the syringe 50. During priming, the air A can pass through the medical fluid to reach the top of the syringe 50 volume adjacent to the plunger 54.

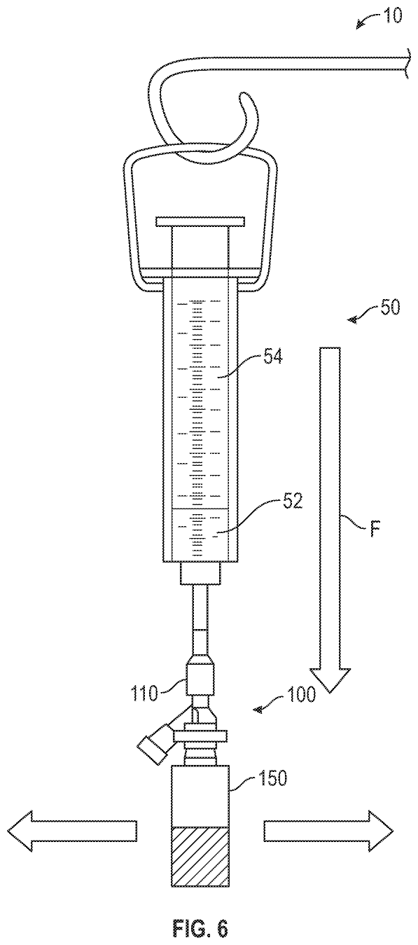

FIG. 6 illustrates the syringe adapter of FIG. 2 with the bulb 150 of the syringe adapter 100 in an expanded configuration. With reference to FIG. 6, as the bulb 150 restores, rebounds, or otherwise expands, the expansion of the bulb volume 152 can draw in medical fluid F into the fluid channel 120 and the bulb volume 152, displacing air that was previously present in the fluid channel 120 and the bulb volume 152. Optionally, the bulb 150 can include a one-way valve to prevent backflow from the IV set from entering the bulb volume 152 during expansion of the bulb volume 152. The one-way valve can be formed from a membrane disposed at a lower portion of the bulb volume 152. During priming, the bulb 150 may be collapsed and expanded multiple times until air is eliminated from the fluid channel 120 and the bulb volume 152. In some embodiments, after priming, the auto-prime cap can be removed from the syringe 50, if utilized, and the syringe adapter 100 can be connected to the patient end of the IV set.

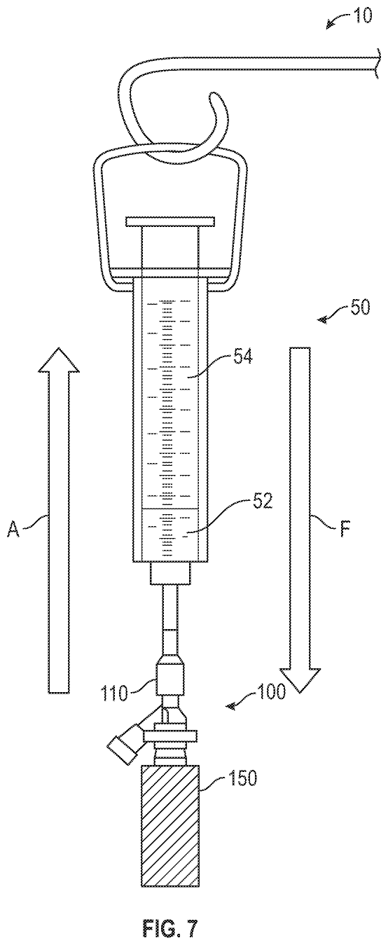

FIG. 7 illustrates the syringe adapter 100 of FIG. 2 with the bulb 150 of the syringe adapter 100 in a primed configuration. With reference to FIG. 7, after the syringe adapter 100 is primed, the bulb volume 152 is filled with medical fluid. Therefore, during operation, medical fluid F from the syringe 50 flows through the fluid channel 120, through the bulb volume 152 and into the IV set. In some embodiments, the bulb volume 152 is in fluid communication with the IV set.

As described above, fluid dispensed by the syringe 50 is displaced by air A, permitting a gravity infusion using a rigid fluid container, such as the syringe 50. Air flow A enters the syringe adapter 100 through the lower portion 124 of the vent channel 122, passes through the check valve 126, through the vent channel 122, through the cannula 140, and into the syringe 50. The air flow A can move to the top of the enclosed syringe 50 volume.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A syringe adapter for coupling with a syringe, the syringe adapter including: an adapter body defining a fluid channel in fluid communication with the syringe and configured to permit medical fluid flow from the syringe through the syringe adapter and a vent channel in fluid communication with the syringe and configured to permit air flow into the syringe; a coupling portion defining an adapter portion configured to be coupled to the adapter body and a syringe portion configured to be coupled to the syringe; and a bulb coupled to the adapter body and defining a bulb volume, wherein the bulb volume is in fluid communication with the fluid channel and is movable between a collapsed configuration configured to displace air from the bulb volume and the fluid channel and an expanded configuration configured to draw medical fluid from the syringe into the fluid channel and bulb volume.
Clause 2. The syringe adapter of Clause 1, wherein the adapter body further defines a spike portion extending from the adapter body.
Clause 3. The syringe adapter of Clause 2, wherein the fluid channel and the vent channel extend through the spike portion of the adapter body.
Clause 4. The syringe adapter of Clause 2, wherein the adapter portion of the coupling portion is configured to be coupled to the spike portion of the adapter body.
Clause 5. The syringe adapter of Clause 1, further including a check valve in fluid communication with the vent channel, wherein the check valve is configured to permit air flow into the syringe and prevent medial fluid flow out of the vent channel.
Clause 6. The syringe adapter of Clause 5, wherein the check valve includes a ball mechanism.
Clause 7. The syringe adapter of Clause 1, further including a cannula extending into the vent channel and configured to extend into the syringe, wherein a cannula lumen of the cannula is in fluid communication with the vent channel.
Clause 8. The syringe adapter of Clause 1, wherein the syringe portion of the coupling portion defines a luer connector configured to couple with the syringe.
Clause 9. A method to prime an IV set, the method including: coupling a syringe to a syringe adapter; displacing air from a fluid channel of the syringe adapter into the syringe by collapsing a bulb coupled to the syringe adapter; and drawing medical fluid from the syringe into the fluid channel of the syringe adapter by expanding the bulb.
Clause 10. The method of Clause 9, further including permitting fluid flow from the syringe to the IV set via the fluid channel.
Clause 11. The method of Clause 10, further including permitting air flow from the environment into the syringe via a vent channel.
Clause 12. An IV set, including: a syringe including: a barrel; and a plunger movably disposed in the barrel, wherein the plunger and the barrel cooperatively defining a syringe volume; and a syringe adapter including: an adapter body defining a fluid channel in fluid communication with the syringe volume and configured to permit medical fluid flow from the syringe volume through the syringe adapter and a vent channel in fluid communication with the syringe volume and configured to permit air flow into the syringe volume; a coupling portion defining an adapter portion coupled to the adapter body and a syringe portion coupled to the syringe; and a bulb coupled to the adapter body and defining a bulb volume, wherein the bulb volume is in fluid communication with the fluid channel and the syringe volume and is movable between a collapsed configuration configured to displace air from the bulb volume and the fluid channel and an expanded configuration configured to draw medical fluid from the syringe volume into the fluid channel and bulb volume.
Clause 13. The IV set of Clause 12, wherein the adapter body further defines a spike portion extending from the adapter body.
Clause 14. The IV set of Clause 13, wherein the fluid channel and the vent channel extend through the spike portion of the adapter body.
Clause 15. The IV set of Clause 13, wherein the adapter portion of the coupling portion is configured to be coupled to the spike portion of the adapter body.
Clause 16. The IV set of Clause 12, further including a check valve in fluid communication with the vent channel, wherein the check valve is configured to permit air flow into the syringe volume and prevent medial fluid flow out of the vent channel.
Clause 17. The IV set of Clause 16, wherein the check valve includes a ball mechanism.
Clause 18. The IV set of Clause 12, further including a cannula extending into the vent channel and configured to extend into the syringe, wherein a cannula lumen of the cannula is in fluid communication with the vent channel.
Clause 19. The IV set of Clause 12, wherein the syringe portion of the coupling portion defines an adapter luer connector.
Clause 20. The IV set of Clause 19, wherein the syringe includes a syringe luer connector configured to couple to the adapter luer connector.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A syringe adapter for coupling with a syringe, the syringe adapter comprising:
an adapter body defining a fluid channel in fluid communication with the syringe and configured to permit medical fluid flow from the syringe through the syringe adapter and a vent channel in fluid communication with the syringe and configured to permit air flow into the syringe;
a coupling portion defining an adapter portion configured to be coupled to the adapter body and a syringe portion configured to be coupled to the syringe; and
a bulb coupled to the adapter body and defining a bulb volume, wherein the bulb volume is in fluid communication with the fluid channel and is movable between a collapsed configuration configured to displace air from the bulb volume and the fluid channel and an expanded configuration configured to draw medical fluid from the syringe into the fluid channel and bulb volume.

2. The syringe adapter of claim 1, wherein the adapter body further defines a spike portion extending from the adapter body.

3. The syringe adapter of claim 2, wherein the fluid channel and the vent channel extend through the spike portion of the adapter body.

4. The syringe adapter of claim 2, wherein the adapter portion of the coupling portion is configured to be coupled to the spike portion of the adapter body.

5. The syringe adapter of claim 1, further comprising a check valve in fluid communication with the vent channel, wherein the check valve is configured to permit air flow into the syringe and prevent medial fluid flow out of the vent channel.

6. The syringe adapter of claim 5, wherein the check valve comprises a ball mechanism.

7. The syringe adapter of claim 1, further comprising a cannula extending into the vent channel and configured to extend into the syringe, wherein a cannula lumen of the cannula is in fluid communication with the vent channel.

8. The syringe adapter of claim 1, wherein the syringe portion of the coupling portion defines a luer connector configured to couple with the syringe.

9. A method to prime an intravenous (IV) set, the method comprising:
coupling a syringe to a syringe adapter;
displacing air from a fluid channel of the syringe adapter into the syringe by collapsing a bulb coupled to the syringe adapter; and
drawing medical fluid from the syringe into the fluid channel of the syringe adapter by expanding the bulb.

10. The method of claim 9, further comprising permitting fluid flow from the syringe to the IV set via the fluid channel.

11. The method of claim 10, further comprising permitting air flow from the environment into the syringe via a vent channel.

12. An intravenous (IV) set, comprising:
a syringe comprising:
a barrel; and
a plunger movably disposed in the barrel, wherein the plunger and the barrel cooperatively defining a syringe volume; and
a syringe adapter comprising:
an adapter body defining a fluid channel in fluid communication with the syringe volume and configured to permit medical fluid flow from the syringe volume through the syringe adapter and a vent channel in fluid communication with the syringe volume and configured to permit air flow into the syringe volume;
a coupling portion defining an adapter portion coupled to the adapter body and a syringe portion coupled to the syringe; and
a bulb coupled to the adapter body and defining a bulb volume, wherein the bulb volume is in fluid communication with the fluid channel and the syringe volume and is movable between a collapsed configuration configured to displace air from the bulb volume and the fluid channel and an expanded configuration configured to draw medical fluid from the syringe volume into the fluid channel and bulb volume.

13. The IV set of claim 12, wherein the adapter body further defines a spike portion extending from the adapter body.

14. The IV set of claim 13, wherein the fluid channel and the vent channel extend through the spike portion of the adapter body.

15. The IV set of claim 13, wherein the adapter portion of the coupling portion is configured to be coupled to the spike portion of the adapter body.

16. The IV set of claim 12, further comprising a check valve in fluid communication with the vent channel, wherein the check valve is configured to permit air flow into the syringe volume and prevent medial fluid flow out of the vent channel.

17. The IV set of claim 16, wherein the check valve comprises a ball mechanism.

18. The IV set of claim 12, further comprising a cannula extending into the vent channel and configured to extend into the syringe, wherein a cannula lumen of the cannula is in fluid communication with the vent channel.

19. The IV set of claim 12, wherein the syringe portion of the coupling portion defines an adapter luer connector.

20. The IV set of claim 19, wherein the syringe comprises a syringe luer connector configured to couple to the adapter luer connector.
